(19) United States
(12) Patent Application Publication
(10) Pub. No.: US 2017/0224683 A1
(43) Pub. Date: Aug. 10, 2017

(54) TREATMENT OF MELANOMA BY BLOCKING BENZAMIL SENSITIVE ION CHANNELS/EXCHANGERS

(71) Applicant: The Board of Trustees of the Leland Stanford Junior University, Stanford, CA (US)

(72) Inventors: M. Peter Marinkovich, Redwood City, CA (US); Carl Gustaf Maarten Winge, Mountain View, CA (US); Atul J. Butte, Menlo Park, CA (US); Mazen Nasrallah, Cambridge, MA (US)

(73) Assignee: The Board of Trustees of the Leland Stanford Junior University, Stanford, CA (US)

(21) Appl. No.: 15/502,457

(22) PCT Filed: Aug. 12, 2015
(86) PCT No.: PCT/US2015/044824

§ 371 Date: Feb. 7, 2017

Related U.S. Application Data
(60) Provisional application No. 62/157,381, filed on May 5, 2015.
(60) Provisional application No. 62/037,518, filed on Aug. 14, 2014.

(51) Int. Cl. A61K 31/4965; A61K 45/06; A61K 9/00

(52) U.S. Cl. CPC A61K 31/4965; A61K 9/0014; A61K 45/06

(57) ABSTRACT

Methods of treatment for melanoma, and compositions for use in such methods are provided. An effective dose of an agent that blocks benzamil-sensitive proteins is administered to an individual suffering from melanoma.

### CROSS REFERENCE

This application is a 371 application and claims the benefit of PCT Application No. PCT/US2015/044824, filed Aug. 12, 2015, which claims benefit of U.S. Provisional Patent Application No. 62/157,381, filed May 5, 2015, and 62/037,518, filed Aug. 14, 2014, which applications are incorporated herein by reference in their entirety.

### GOVERNMENT RIGHTS

This invention was made with Government support under contract AR047223 awarded by the National Institutes of Health. The Government has certain rights in the invention.

### BACKGROUND OF THE INVENTION

Cancer is a leading cause of death worldwide. Melanoma is the most aggressive form of skin cancer. If it is recognized and treated early it is almost always curable, but if it is not, the cancer can advance and spread to other parts of the body, where it becomes hard to treat and can be fatal. While it is not the most common of the skin cancers, it causes the most deaths.

The standard treatment is surgery to remove the tumor and a surrounding area of normal-appearing skin. Sometimes surgery is followed by additional therapy such as immunotherapy, chemotherapy, radiation, or a combination of these treatments. Chemotherapy and immunotherapy are also used to treat advanced or recurrent melanoma. Patients would benefit from new therapies for melanoma, such as those provided by the instant invention.

### SUMMARY OF THE INVENTION

Methods of treatment for melanoma, and compositions for use in such methods are provided. In the methods of the invention, an effective dose of a blocker of a benzamil-sensitive ion channel/exchanger is administered to an individual suffering from melanoma. Blockers of interest include, without limitation, triamterene, amiloride and derivatives, and the like. In some embodiments the blocker is benzyl amiloride (Benzamil). In some instances the dose is effective to reduce metastasis of the cancer.

In some embodiments the blocker is systemically administered to an individual diagnosed with melanoma. In other embodiments the blocker is topically administered, e.g. formulated as a patch, lotion, gel, microneedle array, intralesional injection, etc. to an individual diagnosed with melanoma. The blocker can be formulated in combination with other agents effective in the treatment of melanoma, e.g. standard treatment includes surgery; chemotherapy, e.g. one or more of taxanes, dacarbazine, temozolomide, melphalan, nab-paclitaxel, paclitaxel, carmustine, cisplatin, carboplatin, vinblastine, etc.; radiation therapy; biologic therapy, e.g. with one or more of TNFα, aldesleukin, dabrafenib, interferon alfa-2b, ipilimumab, trametinib, peginterferon alfa-2b , dabrafenib, vemurafenib, oncolytic virus, etc.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A-1C: Benzamil inhibits V12 Rac1 driven melanocyte invasion and proliferation in vitro. In a matrigel invasion assay (48 hours, DMEM, 10% FBS as chemo attractant), V12 Rac1 mutant primary foreskin-derived melanocytes significantly invaded compared to LacZ control. Invasion was abolished in conditions treated with 50 uM Benzamil (FIG. 1A, FIG. 1B). Dosage up to 100 uM were tolerated in vitro (assessed by detectable proliferation), although proliferation-rate was significantly reduced at 50 and 100 uM, (absorbance 570 nanometer; MTT proliferation assay, ATCC, Manassas, US) with and without Benzamil (cultured in DMEM, 10% FBS, 1% antibiotic/antimycotic)(FIG. 1C).

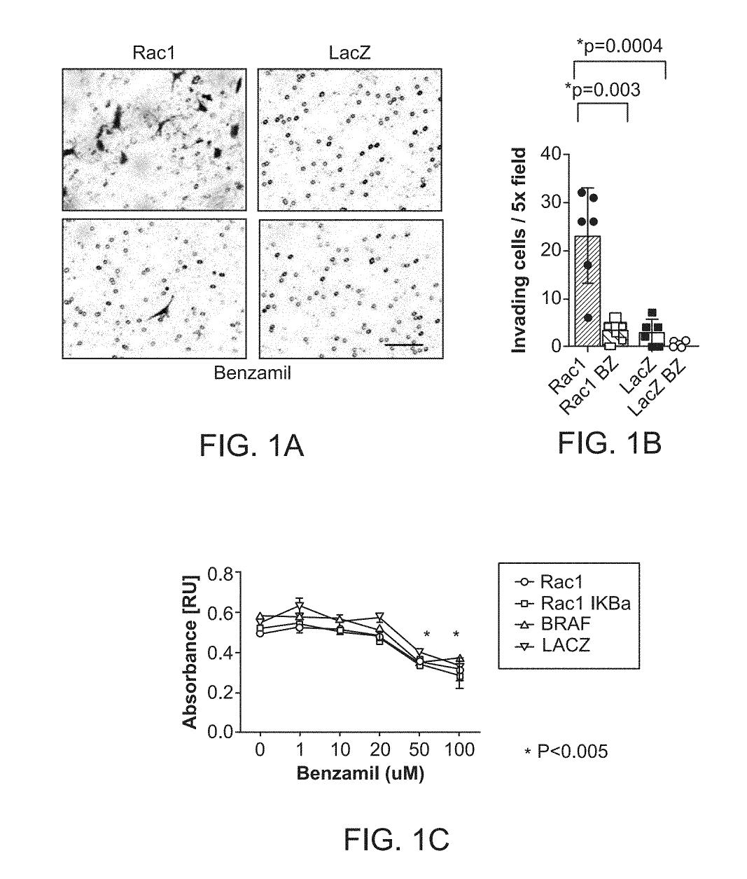

FIG. 2A-2C: Benzamil reduce invasion and lung metastasis in vivo in a xenograft model. V12 Rac1 or LacZ control primary foreskin-derived melanocytes were seeded together with autologous primary keratinocytes ratio 1:4 or 1:10 onto devitalized dermis. After 6 days of culture in KGM media, 10% FBS, 1% antibiotic/antimycotic, skin equivalents were grafted to NOD/SCID gamma mice (n=6). After 10 days sutures and bandages were removed (=day 0), and V12 Rac1 xenografted animals injected IP with Benzamil (5 mg/kg) or PBS every other day for 20 days. Grafts and lungs were harvested on day 21. V12 Rac1 melanocytes displayed primary invasion from the skin (HMB45+ cells sub-GoH3, assayed by confocal IF), distinct from LacZ and significantly more than V12 Rac1 grafts from Benzamil-treated animals FIG. 2A. Moreover, V12 Rac1 grafted animals had multiple foci of HMB45+ cells in lungs FIG. 2B, distinct from control and significantly FIG. 2C more than Benzamil treated animals.

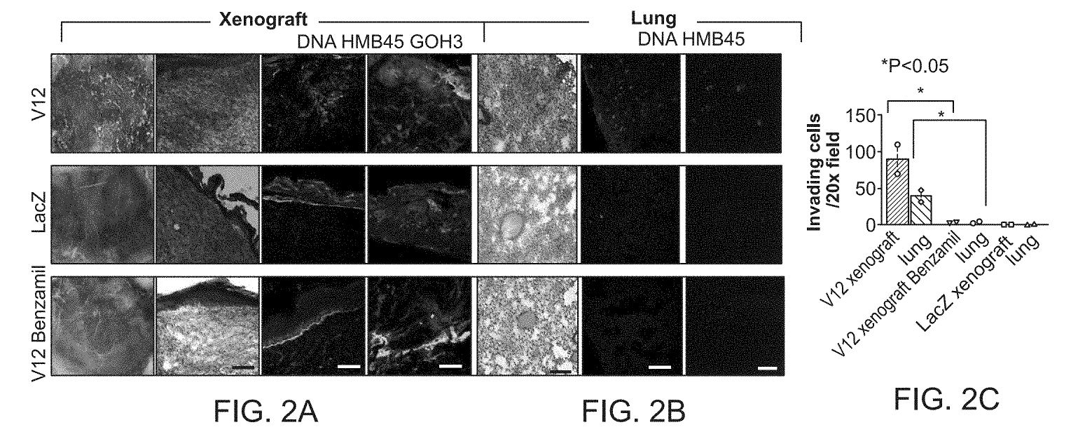

FIG. 3A-3D: Benzamil reduce invasion and proliferation of melanoma cell lines expressing increased Rac1GTP. Melanoma cell lines COLO829, MM485 and CHL1 displayed increased Rac1GTP (Rac1GTP mAb, confocal IF), and 24 hours treatment with Benzamil (50 uM) redistributed Rac1GTP subcellular localization (white arrows) FIG. 3A. Benzamil (50 uM) significantly reduced invasion in matrigel invasion assays of COLO829,MM485,CHL1. Invasion and proliferation was reduced more than PLX-4032 for cell lines MM485 and CHL1 (non-BRAF V600E mutant cell lines), and comparable to a Rac1 inhibitor (Rac1 inhibitor #553502, Calbiochem) (FIG. 3B-3C). Stimulation with Benzamil at 50 uM was not toxic, but reduced proliferation in COLO829,MM485,CHL1 melanoma cell lines similar to V12 Rac1 mutant primary melanocytes FIG. 3D.

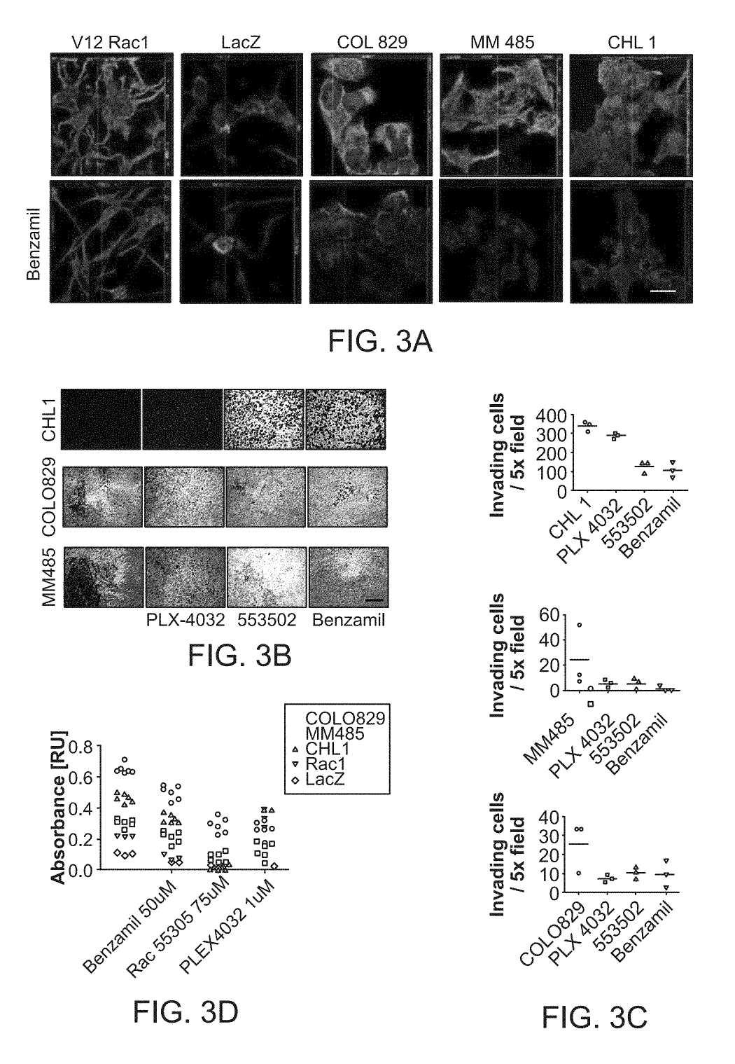

FIG. 4A-4C: Reduction of NFKB (PRELA) and STAT3 (PSTAT3) signaling following Benzamil treatment in vivo and in vitro. FIG. 4A; Skin from V12 Rac1, control or Benzamil treated V12 Rac1 xenografted animals were assayed by confocal IF for phospho-Rela (upper) and phospo-STAT3 (lower panel) co-localizing with HMB45+ melanocytes. Both NFKB and STAT3 activation in HMB45+ cells was reduced in skin following benzamil treatment (5 mg/kg; 10 injections, EOD). Nuclear translocation of activated STAT3 FIG. 4B; phosphorylation, nuclear translocation) and RelA FIG. 4C; phosphorylation, nuclear translocation) in melanoma cell lines CHL1, COLO829 and MM485 was assayed after 24 hours by confocal IF of methanol-fixed cells on collagen coated coverslips, with and without exposure to Benzamil (50 μM). STAT3 and NFKB activation was diminished following Benzamil treatment for 24 hours in all three cell lines.

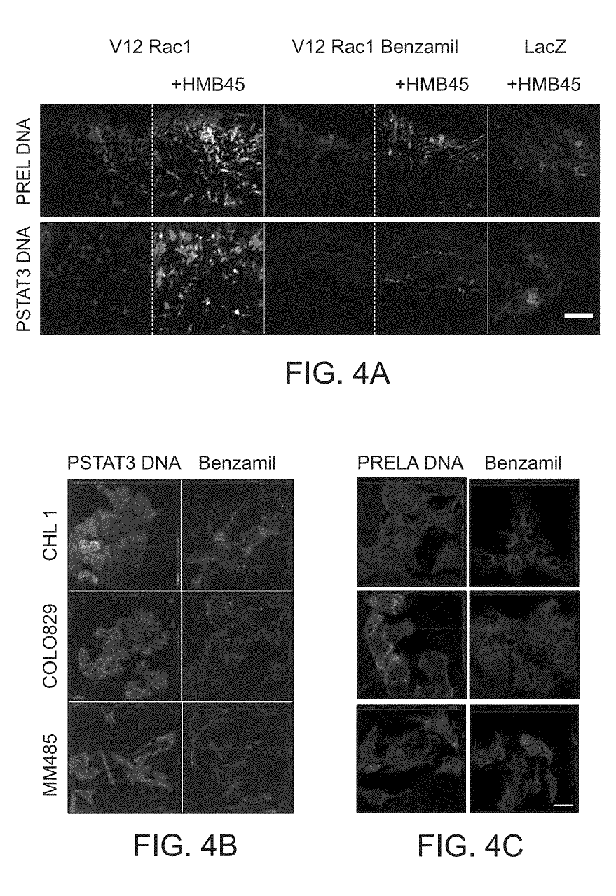

FIG. 5A-5B: Benzamil prevents V12 Rac1 and BRAF V600E driven invasion in vivo. One week after SQ-injection of 500 000 luciferase-infected Rac1MCs, COLO829 (V600E mutant) or MM485 (NRAS Q61L mutant) melanocytes into NOD/SCID mice (n=3 per group), mice of equal tumor size (by bioluminescence) were either treated with Benzamil (10 mg/kg) or vehicle IP daily for weeks. Benzamil treatment reduced primary tumor growth and prevented invasion into underlying tissue of both Rac1MC and COLO829 melanocytes. Representative bioluminescence IVIS image of mice treated with with vehicle (−) or Benzamil (+) shown FIG. 5A. Quantification of pixel intensity and primary tumor weight FIG. 5B. Hematoxylin & eosin staining black arrowheads; Confocal IF white arrowheads, HMB45-green, Ki67-red, DNA-blue.

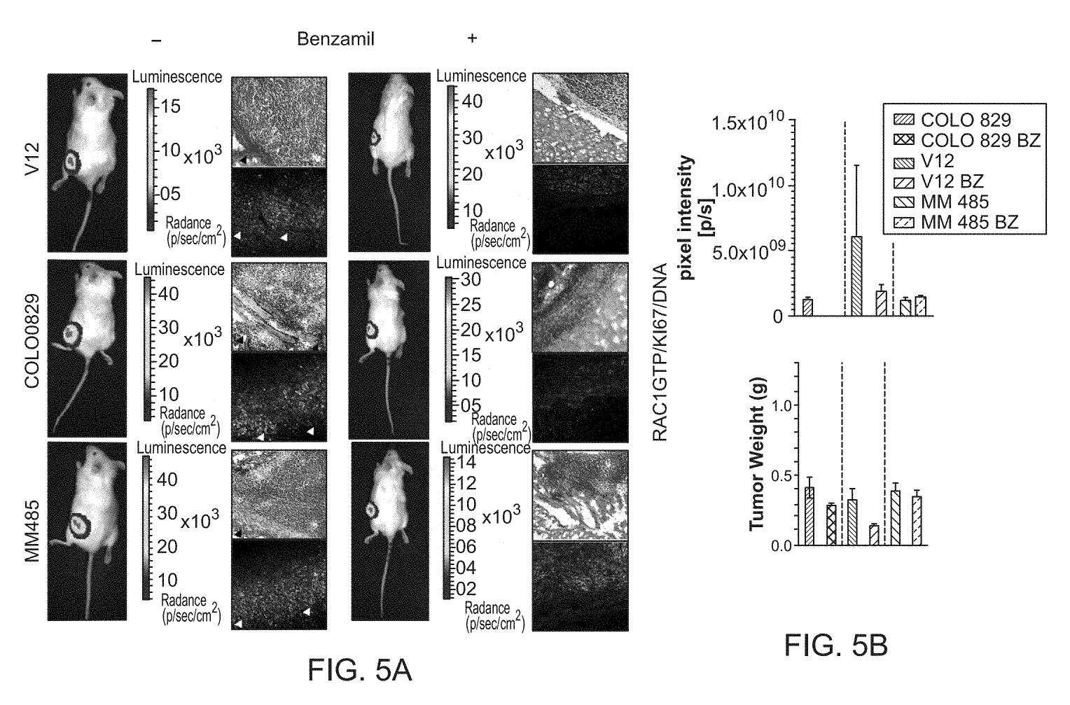

FIG. 6A-6B: Benzamil prevents V12 Rac1 and BRAF V600E metastasis in vivo. After 5 weeks of Benzamil or vehicle IP of V12, COLO829 or MM485 SQ-injected, luciferase-infected melanoma cells, lungs were isolated ex-vivo (n=3 per group), and bioluminescence assayed and quantified prior to histological analysis (hematoxylin & eosin) and confocal microscopy of indirect IF. Representative bioluminescence IVIS images of lungs (2) per group, treated with vehicle (−) or Benzamil (+) are depicted. Benzamil-treated V12 and COLO829 mice showed significantly reduced lung metastasis (FIG. 6A, FIG. 6B). HMB45-green, Ki67-red, DNA-blue.

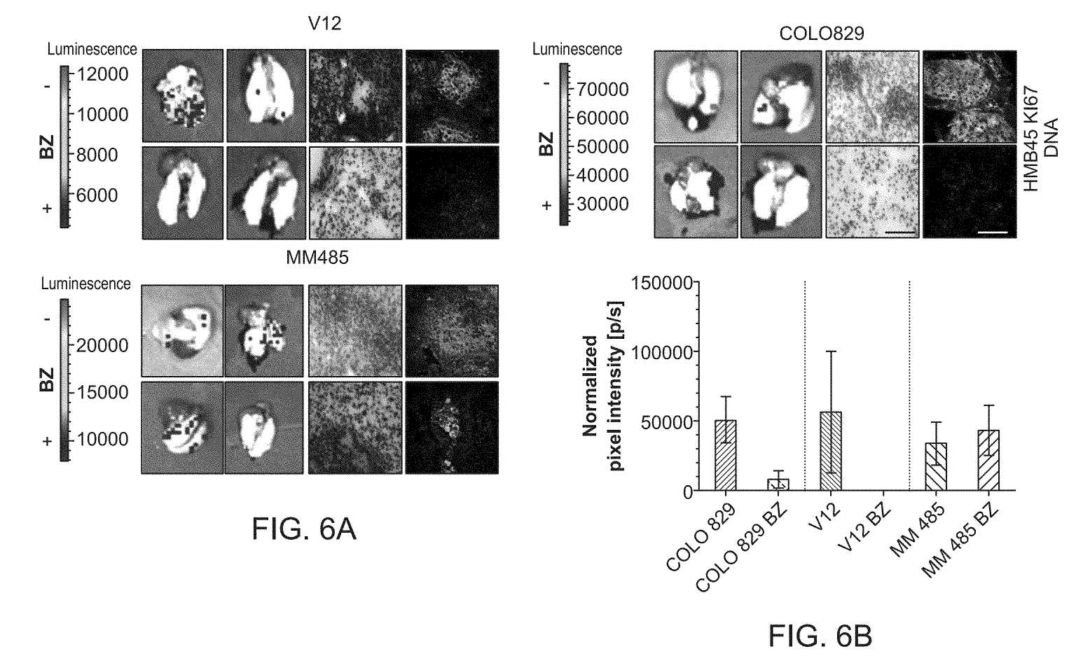

FIG. 7A-7B: A xenograft model of V12 Rac1 driven invasion from skin. Organotypic skin equivalents harboring primary V12Rac1 (n=4)or LacZ MCs (n=2) epidermally seeded with keratinocytes (1:32) and dermally seeded fibroblasts (500 000 per graft) for 7 days at air-fluid interphase, were xenografted to NOD/SCID mice. Ten days after grafting, v12 mice were treated with benzamil IP (10 mg/kg, n=2) or vehicle (n=2) for 4 weeks. Controls demonstrated invasion from graft, (FIG. 7A, FIG. 7B), however Benzamil treatment showed strongly reduced invasion (dermal presence of HMB-45+ cells). Middle panels: HMB45-red, GOH-green, DNA blue, right panel P65-red, HMB45-green, DNA-blue; far right phopsho-p65-red, DNA-blue).

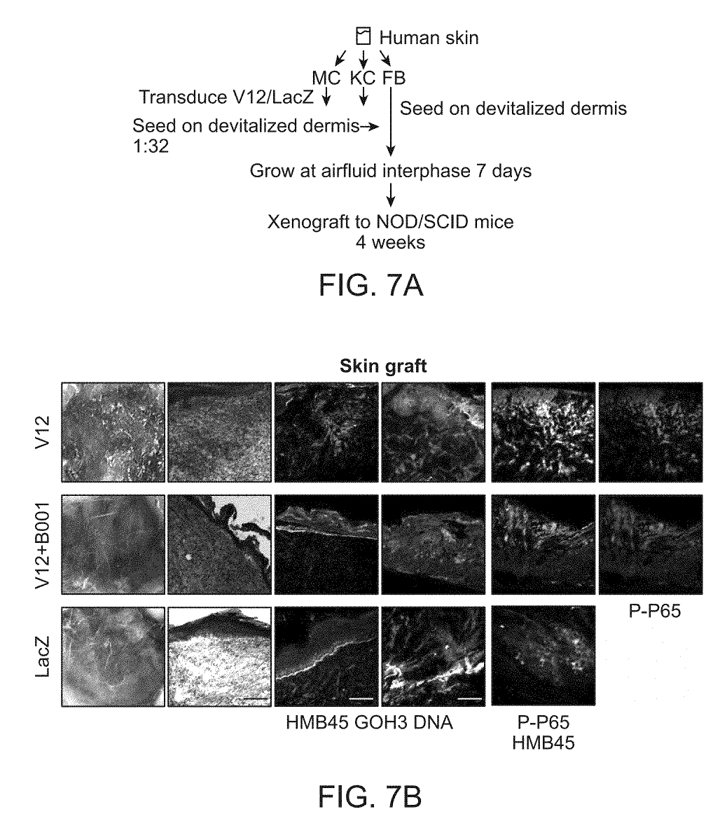

FIG. 8A-8B: A xenograft model of V12 Rac1 driven metastasis from skin. Lungs from mice carrying V12Rac1 MC xenografts (top) had a dense cell infiltrate (hematoxylin & eosin) and widespread melanoma cells in lungs (HMB45+ ), whereas Benzamil-treated V12Rac1 MC xenograft-mice (middle) had markedly reduced cell infiltrates, comparable to control (LacZ), and strongly diminished HMB45+ cells in lungs compared to vehicle-treated V12Rac1 xenograft mice. Quantification of HMB45+ cells per 20× field showed significant reduction in V12MCs metastasizing to lungs. HMB45-red, DNA-blue.

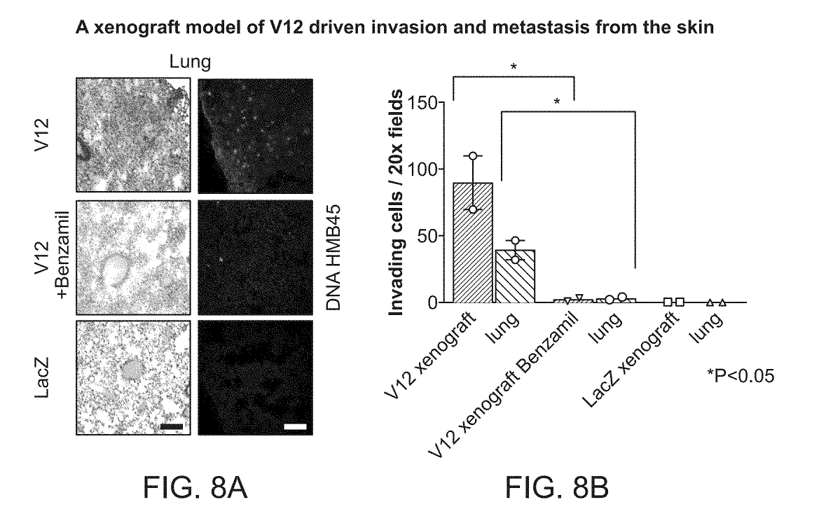

### DETAILED DESCRIPTION OF THE EMBODIMENTS

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, illustrative methods, devices and materials are now described.

All publications mentioned herein are incorporated herein by reference for the purpose of describing and disclosing the subject components of the invention that are described in the publications, which components might be used in connection with the presently described invention.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

The term “tumor,” as used herein, refers to neoplastic cell growth and proliferation, whether malignant or benign, and all pre-cancerous and cancerous cells and tissues.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. In general, cells of interest for detection, analysis, classification, or treatment in the present application include precancerous (e.g., benign), malignant, pre-metastatic, metastatic, and non-metastatic cells.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body. Therefore, the present invention contemplates a method of determining the risk of further growth of one or more cancerous tumors in an organ or body part which is not directly connected to the organ of the original cancerous tumor and/or any steps in a process leading up to that growth.

Melanoma. Malignant melanoma is a cancer that arises from melanocytes in a pigmented area (eg, skin, mucous membranes, eyes, or CNS). Metastasis is correlated with depth of dermal invasion. With spread, prognosis is poor. Melanoma accounts for <5% of total skin cancers diagnosed in the US but causes most skin cancer deaths.

Melanomas occur mainly on the skin but also on the mucosa of the oral, genital, and rectal regions and conjunctiva. Melanomas may also develop in the choroid layer of the eye and in the nail beds. Melanomas vary in size, shape, and color (usually pigmented) and in their propensity to invade and metastasize. Metastasis occurs via lymphatics and blood vessels. Local metastasis results in the formation of nearby satellite papules or nodules that may or may not be pigmented. Metastasis to skin or internal organs may occur, and, occasionally, metastatic nodules or enlarged lymph nodes are discovered before the primary lesion is identified.

About 30% of melanomas develop from pigmented mole; almost all the rest arise from melanocytes in normal skin. Atypical moles (dysplastic nevi) may be precursors to melanoma. The very rare melanomas of childhood almost always arise from giant congenital nevi present at birth.

Superficial spreading melanoma accounts for about 70% of cases. Typically asymptomatic, it occurs most commonly on women's legs and men's torsos. The lesion is usually a plaque with irregular, raised, indurated, and tan or brown areas, which often have red, white, black, and blue spots or small, sometimes protuberant blue-black nodules. Small notchlike indentations of the margins may be noted, along with enlargement or color change. Histologically, atypical melanocytes characteristically invade the dermis and epidermis. This type of melanoma most commonly has activating mutations in the BRAF gene at V600.

Nodular melanoma accounts for 15 to 30% of melanomas. It may occur anywhere on the body as a dark, protuberant papule or a plaque that varies from pearl to gray to black. Occasionally, a lesion contains little if any pigment or may look like a vascular tumor. Unless it ulcerates, nodular melanoma is asymptomatic, but patients usually seek advice because the lesion enlarges rapidly.

Lentigo maligna melanoma accounts for 5% of melanomas. It tends to arise in older patients. It arises from lentigo maligna. It usually occurs on the face or other areas of chronic sun exposure as an asymptomatic, flat, tan or brown, irregularly shaped macule or patch with darker brown or black spots scattered irregularly on its surface. In lentigo maligna, both normal and malignant melanocytes are confined to the epidermis. When malignant melanocytes invade the dermis, the lesion is called lentigo maligna melanoma, and the cancer may metastasize. This type of melanoma most commonly has mutations in the C-kit gene.

Acral-lentiginous melanoma accounts for only 2 to 10% of melanomas. Incidence is probably the same regardless of skin pigmentation, but because people with darkly pigmented skin infrequently develop other forms of melanoma, acral-lentiginous melanoma is the most common type among them. It arises on palmar, plantar, and subungual skin and has a characteristic histologic picture similar to that of lentigo maligna melanoma. This type of melanoma often has mutations in the C-kit gene.

The staging of melanoma is based on clinical and pathologic criteria and closely corresponds to the traditional tumor-node-metastasis (TNM) classification system. The staging system classifies melanomas based on local, regional, or distant disease. Stages I and II: Localized primary melanoma. Stage III: Metastasis to regional lymph nodes. Stage IV: Distant metastatic disease. Stage strongly correlates with survival. A minimally invasive microstaging technique, the so-called sentinel lymph node biopsy (SLNB), is a major advance in the ability to stage cancers more accurately. Recommended staging studies depend on the Breslow depth (how deeply tumor cells have invaded) and histologic characteristics of the melanoma; dermal mitoses and ulceration indicate higher risk in melanomas that are <1 mm Breslow depth. Staging studies may include SLNB, laboratory tests (eg, CBC, LDH, liver function tests), chest x-ray, CT, and PET and are done by a coordinated team that includes dermatologists, oncologists, general surgeons, plastic surgeons, and dermatopathologists.

Melanoma may spread rapidly, causing death within months of its recognition, yet the 5-yr cure rate of early, very superficial lesions is very high. Thus, cure depends on early diagnosis and early treatment. For tumors of cutaneous origin (not CNS and subungual melanomas) that have not metastasized, the survival rate varies depending on the thickness of the tumor at the time of diagnosis. Once melanoma has metastasized to the lymph nodes, 5-yr survival ranges from 25 to 70% depending on the degree of ulceration and number of nodes involved. Once melanoma has metastasized to distant sites, 5-yr survival is about 10%.

Conventional treatment is primarily by surgical excision (wide local excision). A 1-cm lateral tumor-free margin is generally adequate for lesions <1 mm thick. In tumors <1 mm thick, but with ulceration or at least 1 dermal mitoses/mm2, SLNB can be considered. Thicker lesions may deserve larger margins, more radical surgery, and SLNB. Lentigo maligna melanoma and lentigo maligna are usually treated with wide local excision and, if necessary, skin grafting.

Metastatic disease is generally inoperable, but in certain cases, localized and regional metastases can be excised. Conventional treatment includes chemotherapy with dacarbazine or temozolamide (oral dacarbazine analog) and aldesleukin can be used for the treatment of metastatic melanoma. Adjuvant therapy with recombinant biologic response modifiers (particularly interferon alfa) to suppress clinically inapparent micrometastases may also be used for inoperable metastatic melanoma. Brain metastases may be treated with palliative radiation, but the response is poor. Ipilimumab (a monoclonal antibody to cytotoxic T lymphocyte-associated antigen 4 [CTLA-4]) is now available for unresectable or metastatic melanoma, for which it is sometimes now considered the treatment of choice. Vemurafenib (a BRAF inhibitor) is a treatment of choice for unresectable or metastatic melanomas that have the V600 E BRAF mutation. It works by inhibiting BRAF activity, resulting in slowing or stopping of tumor cell proliferation. Nivolumab and labrolizumab are investigational antibodies that inhibit the programmed death (PD-1) receptor that attenuates T-cell effector responses against cancers.

Benzamil sensitive proteins. Benzamil and other amiloride-analogues act at two large classes of proteins. The first are members of the Degenerin/Epithelial Sodium Channel (Deg/ENaC) Superfamily of ion channels. All family members share a common topology, with subunits containing two membrane-spanning regions and a large, cysteine-rich extracellular loop. Mammalian family members include the ENaCs and the acid sensitive ion channels, or ASICs. In both groups, the proteins form multimers composed of four subunits. Two subunits, which are the main structural components of the channel, are repeated. The remaining subunits are modulatory or accessory components of the channel.

ENaCs are sodium channels, and are involved in salt homeostasis. ENaCs play a critical role in sodium reabsorption in the distal nephron, as well as at the lung and colon. ENaCs may also play a role in the arterial baroreceptor reflex. A 6 subunit, sharing much homology with the oc subunit, has been identified. Although 6 ENaC has a broad neural distribution, to date, it has only been found in primates.

The ASICs are proton gated, non-selective cation channels, which are widely expressed in neural tissue. ASICs play a role in such diverse functions as nociception, response to ischemic events, and ability to taste salt. Gating properties and channel function are dramatically affected by the particular subunits present, and so the specific multimer formed may dictate the physiological function of the channel.

The second large class of benzamil-sensitive proteins are ion transport systems. These include the Na⁺/H⁺ exchanger, the Na⁺/Ca⁺⁺ exchanger, the Na⁺ pump, and the Ca⁺⁺ pump. The Na⁺/H⁺ ion exchanger, or antiport, is a membrane-localized protein found in a wide variety of cell types. It relies on secondary active transport to facilitate the movement of ions (i.e., ion flux generated by active transport at other proteins, such as the Na⁺/K⁺ ATPase generate the gradients needed to run these ion exchangers). Increased activity of the Na⁺/H⁺ exchanger has been linked to primary hypertension in humans.

The Na⁺/Ca⁺⁺ exchanger is a bidirectional transporter, whose activity depends on the electrochemical gradients present at the membrane. The Na⁺/Ca⁺⁺ exchanger's activity is coupled to that of the Na⁺/H⁺ antiport, and may also play a role in hypertension. While amiloride and benzamil are inhibitors of these proteins, the ion transport systems have higher affinity for other amiloride analogues, such as 3′,4′-dichlorobenzamil; 2′,4′-dimethylbenzamil; 5-(N-ethyl-N-isopropyl) amiloride; and 5-(N-methyl-N-isobutyl) amiloride.

Blocker. A blocker of the invention targets a benzamil sensitive protein, as described above, and competes with benzamil as a blocker. Known blockers include triamterene, phenamil, amiloride and amiloride derivatives, particularly benzyl amiloride (Benzamil), 3′,4′-dichlorobenzamil; 2′,4′-dimethylbenzamil; 5-(N-ethyl-N-isopropyl) amiloride; and 5-(N-methyl-N-isobutyl) amiloride. Additional amiloride derivatives are described in WO2012035158; WO2009074575; WO2011028740; WO2009150137; WO2011079087; and WO2008135557, each of which are herein specifically incorporated by reference. In one embodiment, benzamil is used in the methods and compositions of the invention.

As used herein, and unless otherwise specified, the term “therapeutically effective amount” of a compound is an amount sufficient to provide a therapeutic benefit in the treatment or management of melanoma, or to delay or minimize one or more symptoms associated with melanoma. A therapeutically effective amount of a compound means an amount of therapeutic agent, alone or in combination with other therapies, which provides a therapeutic benefit in the treatment or management of melanoma. The term “therapeutically effective amount” can encompass an amount that improves overall therapy, reduces or avoids symptoms or causes of melanoma, or enhances the therapeutic efficacy of another therapeutic agent. The therapeutically effective dose may be sufficient to reduce or prevent metastasis of melanoma.

The effective dosage range for the administration of the blocker depends upon the form of the blocker and its potency. It is an amount large enough to produce the desired effect in which symptoms of melanoma are ameliorated (e.g. inhibition of tumor growth, inhibition of metastasis, etc.). The phrase “therapeutically-effective amount” as used herein means that amount of blocker or composition comprising the blocker which is effective for producing the desired therapeutic effect, in at least a sub-population of cells, in a subject at a reasonable benefit/risk ratio applicable to any medical treatment. For example, an amount of a compound administered to a subject that is sufficient to produce a statistically significant, measurable change in at least one symptom of melanoma. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents. There are preclinical melanoma models that are well known to those of skill in the art which can be used to determine therapeutically effective amounts of the compound or agents and to optimize administration regimes.

In one embodiment, a therapeutically effective amount of blocker inhibits melanoma tumor volume or metastasis in a preclinical model by at least 5%, at least 10%, at least 20%, at least 30%, at least 40%, at least 50%, at least 60%, at least 70%, at least 80%, at least 90% and reduces at least one symptom of melanoma by at least 5%, at least 10%, at least 20%, at least 30%, at least 40%, at least 50%, at least 60%, at least 70%, at least 80%, or at least 90%. For example, tumor volumes in xenograft mice can be calculated using the following ellipsoid formula: [Dx(d2)]/2, in which D represents the large diameter of the tumor, and d represents the small diameter. Tumor volumes of treated groups are presented as percentages of tumor volumes of the control groups (% T/C) using the following formula: 100×[(T−T₀)/(C−C₀)], in which T represents mean tumor volume of a treated group on a specific day during the experiment, T₀ represents mean tumor volume of the same treated group on the first day of treatment, C represents mean tumor volume of a control group on the specific day during the experiment, and C₀ represents mean tumor volume of the same treated group on the first day of treatment. Percent tumor growth inhibition can be calculated as 100-% T/C, with >100% tumor growth inhibition representing regression. Survival can be calculated using a predefined cutoff volume of 2,000 mm³ as a surrogate for mortality.

In one embodiment a therapeutically effective amount of the blocker inhibits cellular proliferation of melanoma cells in a preclinical model by at least 5%, at least 10%, at least 20%, at least 30%, at least 40%, at least 50%, at least 60%, at least 70%, at least 80%, or at least 90% and reduces at least one symptom of melanoma by at least 5%, at least 10%, at least 20%, at least 30%, at least 40%, at least 50%, at least 60%, at least 70%, at least 80%, or at least 90%. Inhibition of cellular proliferation may be evaluated by 3-(4,5-dimethylthiazole-2-yl)-2,5-diphenyl-2H-tetrazolium bromide (MTT; Sigma) assay. For example cells can be plated in 96-well microtiter plates at a density of 1,000 to 5,000 cells per well in a volume of 180 μl. Twenty-four hours after cell plating, 20 μl of an appropriate compound/agent dilution can be added to plates in duplicate. The plates may then be assayed for proliferation 6 days after the cells were plated according to known methods in the art. Percent inhibition can then be calculated and the IC₅₀ determined from the regression of a plot of the logarithm of the concentration versus percent inhibition.

The therapeutically effective dose can be estimated initially from a suitable cell culture or enzymatic assays (e.g. melanoma cell growth assays, etc.), then a dose of each compound and treatment regime may be formulated in animal models to achieve a circulating plasma concentration range that includes the IC₅₀ as determined in cell culture.

For administration to a subject, the compounds or agents can be provided in pharmaceutically acceptable compositions. These pharmaceutically acceptable compositions comprise a therapeutically-effective amount of one or more blockers, formulated together with one or more pharmaceutically acceptable carriers (additives) and/or diluents. The pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally (e.g. as a nasal spray or suppository); or (9) nasally. Additionally, compounds can be implanted into a patient or injected using a drug delivery system. Guidance for formulations can be found in e.g. Remington: The Science and Practice of Pharmacy by Alfonso R. Gelmaro (Ed.) 20.sup.th edition: Dec. 15, 2000, Lippincott, Williams & Wilkins, ISBN: 0683306472

As used herein, the term “dose amount” refers to the quantity, e.g., milligrams (mg), of the substance which is administered to the subject. In one embodiment, the dose amount is a fixed dose, e.g., is not dependent on the weight of the subject to which the substance is administered. In another embodiment, the dose amount is not a fixed dose, e.g., is dependent on the weight of the subject to which the substance is administered, or for a topical therapy a dose may be related to the surface area that is treated, e.g. dose/m² of skin.

Exemplary dose amounts, e.g., fixed dose amounts, for use treating an adult human by the methods of the invention include, about 0.01 mg, about 0.05 mg, about 0.1 mg, about 0.5 mg, about 1 mg, about 5 mg, about 10 mg, about 50 mg, about 100 mg, about 500 mg, or more.

Exemplary dose amounts, e.g., dose amounts for topical use treating an adult human by the methods of the invention include, about 0.01 mg/m² surface area, about 0.05 mg/m² surface area, about 0.1 mg/m² surface area, about 0.5 mg/m² surface area, about 1 mg/m² surface area, about 5 mg/m² surface area, about 10 mg/m² surface area, about 50 mg/m² surface area, about 100 mg/m² surface area, about 500 mg/m² surface area, or more.

Ranges intermediate to the above-recited ranges are also contemplated by the invention. For example, ranges having any one of these values as the upper or lower limits are also intended to be part of the invention, e.g., about 0.01 mg to about 100 mg, about 1 mg to about 10 mg, etc.

Dosage Form: As used herein, the term “dosage form” refers to a physically discrete unit of an active agent (e.g., a therapeutic or diagnostic agent) for administration to a subject. Each unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing Regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

As used herein, the term “periodicity” as it relates to the administration of a substance refers to a (regular) recurring cycle of administering the substance to a subject. In one embodiment, the recurring cycle of administration of the substance to the subject achieves a therapeutic objective. The periodicity of administration of the substance may be about once a week, once every other week, about once every three weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, about once every 20 weeks, about once every 21 weeks, about once every 22 weeks, about once every 23 weeks, about once every 24 weeks, about once every 5-10 days, about once every 10-20 days, about once every 10-50 days, about once every 10-100 days, about once every 10-200 days, about once every 25-35 days, about once every 20-50 days, about once every 20-100 days, about once every 20-200 days, about once every 30-50 days, about once every 30-90 days, about once every 30-100 days, about once every 30-200 days, about once every 50-150 days, about once every 50-200 days, about once every 60-180 days, or about once every 80-100 days. Periodicities intermediate to the above-recited times are also contemplated by the invention. Ranges intermediate to the above-recited ranges are also contemplated by the invention. For example, ranges having any one of these values as the upper or lower limits are also intended to be part of the invention, e.g., about 110 days to about 170 days, about 160 days to about 220 days, etc.

The “duration of a periodicity” refers to a time over which the recurring cycle of administration occurs. For example, a duration of the periodicity of administration of a substance may be may be up to about 4 weeks, up to about 8 weeks, up to about 12 weeks, up to about 16 weeks or more, up to about 20 weeks, up to about 24 weeks, up to about 28 week, up to about 32 weeks or more, during which the periodicity of administration is about once every week. For example, a duration of the periodicity may be about 6 weeks during which the periodicity of administration is about once every 4 weeks, e.g., the substance is administered at week zero and at week four.

The term “likelihood” generally refers to an increase in the probability of an event. The term “likelihood” when used in reference to the effectiveness of a patient response generally contemplates an increased probability that the symptoms of melanoma will be lessened or decreased.

The terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

The term “sample” as used herein relates to a material or mixture of materials, typically, although not necessarily, in fluid form, containing one or more components of interest. “Biological sample” as used herein refers to a sample obtained from a biological subject, including sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, fractions and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include but are not limited to whole blood, partially purified blood. PBMCs, tissue biopsies, and the like.

Comparable: As used herein, the term “comparable” refers to two or more agents, entities, situations, sets of conditions, etc., that may not be identical to one another but that are sufficiently similar to permit comparison there between so that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: A “composition” or a “pharmaceutical composition” according to this invention refers to an agent (e.g. amiloride, benzamil, etc.) or combination of two or more agents as described herein for co-administration or administration as part of the same regimen. It is not required in all embodiments that the combination of agents result in physical admixture, that is, administration as separate co-agents each of the components of the composition is possible; however practitioners in the field may find it advantageous to prepare a composition that is an admixture of two or more of the ingredients in a pharmaceutically acceptable carrier, diluent, or excipient, making it possible to administer the component ingredients of the combination at the same time.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of” ) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of” ) the named elements or steps to the exclusion of any other unnamed element or step. In any composition or method disclosed herein, known or disclosed equivalents of any named essential element or step may be substituted for that element or step.

Determine: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Patient: As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the patient is receiving or has received certain therapy to diagnose and/or to treat a disease, disorder, or condition.

As used herein, “treatment” is an approach for obtaining beneficial or desired clinical results. Beneficial or desired clinical results may include, but are not limited to, any one or more of: alleviation of one or more symptoms, diminishment of extent of disease, stabilized (i.e., not worsening) state of disease, preventing or delaying spread (e.g., metastasis) of disease, preventing or delaying occurrence or recurrence of disease, delay or slowing of disease progression, amelioration of the disease state, and remission (whether partial or total). Also encompassed by “treatment” is a reduction of pathological consequence of a proliferative disease such as cancer (e.g., melanoma). The methods provided herein contemplate any one or more of these aspects of treatment.

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “kit” as used herein refers to a packaged product comprising components with which to administer the blocker of the invention for treatment of melanoma. The kit preferably comprises a box or container that holds the components of the kit. The box or container may be affixed with a label or a Food and Drug Administration approved protocol. The box or container holds components of the invention which are preferably contained within plastic, polyethylene, polypropylene, ethylene, or propylene vessels. The vessels can be capped-tubes or bottles. The kit can also include instructions for use.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The amount of compound which can be combined with a carrier material to produce a single dosage form will generally be that amount of the compound which produces a therapeutic effect. Generally out of one hundred percent, this amount will range from about 0.1% to 99% of compound, preferably from about 5% to about 70%, most preferably from 10% to about 30%.

Formulations suitable for parenteral administration conveniently include sterile aqueous preparation of the active compound which is preferably isotonic with the blood of the recipient. Thus, such formulations may conveniently contain distilled water, 5% dextrose in distilled water or saline. Useful formulations also include concentrated solutions or solids containing the compound which upon dilution with an appropriate solvent give a solution suitable for parental administration above.

For enteral administration, a compound can be incorporated into an inert carrier in discrete units such as capsules, cachets, tablets or lozenges, each containing a predetermined amount of the active compound; as a powder or granules; or a suspension or solution in an aqueous liquid or non-aqueous liquid, e.g., a syrup, an elixir, an emulsion or a draught. Suitable carriers may be starches or sugars and include lubricants, flavorings, binders, and other materials of the same nature.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine, a mixture of the powdered active compound with any suitable carrier.

A syrup or suspension may be made by adding the active compound to a concentrated, aqueous solution of a sugar, e.g., sucrose, to which may also be added any accessory ingredients. Such accessory ingredients may include flavoring, an agent to retard crystallization of the sugar or an agent to increase the solubility of any other ingredient, e.g., as a polyhydric alcohol, for example, glycerol or sorbitol.

Formulations for rectal administration may be presented as a suppository with a conventional carrier, e.g., cocoa butter or Witepsol S55 (trademark of Dynamite Nobel Chemical, Germany), for a suppository base.

Formulations for oral administration may be presented with an enhancer. Orally-acceptable absorption enhancers include surfactants such as sodium lauryl sulfate, palmitoyl carnitine, Laureth-9, phosphatidylcholine, cyclodextrin and derivatives thereof; bile salts such as sodium deoxycholate, sodium taurocholate, sodium glycochlate, and sodium fusidate; chelating agents including EDTA, citric acid and salicylates; and fatty acids (e.g., oleic acid, lauric acid, acylcarnitines, mono- and diglycerides). Other oral absorption enhancers include benzalkonium chloride, benzethonium chloride, CHAPS (3-(3-cholamidopropyl)-dimethylammonio-1-propanesulfonate), Big-CHAPS(N,N-bis(3-D-gluconamidopropyl)-cholamide), chlorobutanol, octoxynol-9, benzyl alcohol, phenols, cresols, and alkyl alcohols. An especially preferred oral absorption enhancer for the present invention is sodium lauryl sulfate.

As used herein, the term “administer” or “administering” refers to the placement of a composition into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

An alternative formulation for topical delivery is an array of microneedles. Microneedles (MN), as used herein, refers to an array comprising a plurality of micro-projections, generally ranging from about 25 to about 2000 μm in length, which are attached to a base support. An array may comprise 10², 10³, 10⁴, 10⁵ or more microneedles, and may range in area from about 0.1 cm² to about 100 cm². Application of MN arrays to biological membranes creates transport pathways of micron dimensions, which readily permit transport of macromolecules such as large polypeptides. In some embodiments of the invention, the microneedle array is formulated as a transdermal drug delivery patch. MN arrays can alternatively be integrated within an applicator device which, upon activation, can deliver the MN array into the skin surface, or the MN arrays can be applied to the skin and the device then activated to push the MN through the SC.

Various materials have been used for microneedles. For example, biodegradable materials into which the therapeutic agent, e.g. Benzamil, can be incorporated are of interest. Such materials include various biodegradable or biocompatible polymers or cross-linked monomers, as known in the art. The dose of agent to be delivered will vary, and may range from at least about 1 ng/microneedle array, at least about 10 ng, at least about 0.1 μg, at least about 1 μg, at least about 10 μg, at least 0.1 mg, at least 1 mg, or more in a single array. MNs may be fabricated with a wide range of designs (different sizes and shapes) and different types (solid, hollow, sharp, or flat), and may be in-plane and/or out-of-plane.

Polymeric MNs can provide biocompatibility, biodegradability, strength, toughness, and optical clarity. To accurately produce the micro-scale dimensions of polymer MNs, a variety of mould-based techniques, such as casting, hot embossing, injection molding, and investment molding may be used, e.g. beveled-tip, chisel-tip, and tapered-cone polydimethylsiloxane (PDMS) molds. Polymeric materials of interest for fabrication include without limitation; poly (methylmetha-acrylate) (PMMA), poly-L-lactic acid (PLA), poly-glycolic acid (PGA), and poly-lactic-co-glycolic acid (PLGA), cyclic-olefin copolymer, poly (vinyl pyrrolidone), and sodium carboxymethyl cellulose. Sugars have also been used to fabricate the MNs, such as galactose, maltose, aliginate, chitosan, and dextrin. Materials may be cross-linked through ion exchange, photo-polymerization, and the like.

In other embodiments, a topical formulation is provided as a transdermal patch. Medical dressings suitable for formulation in a transdermal patch can be any material that is biologically acceptable and suitable for placing over the skin. In exemplary embodiments, the support may be a woven or non-woven fabric of synthetic or non-synthetic fibers, or any combination thereof. The dressing may also comprise a support, such as a polymer foam, a natural or man-made sponge, a gel or a membrane that may absorb or have disposed thereon, a therapeutic composition. A gel suitable for use as a support is sodium carboxymethylcellulose 7H 4F, i.e. ethylcellulose.

For example, hydrocolloids (eg, RepliCare, DuoDERM, Restore, Tegasorb), which are combinations of gelatin, pectin, and carboxymethylcellulose in the form of wafers, powders, and pastes; some have adhesive backings and others are typically covered with transparent films to ensure adherence. Alginates (polysaccharide seaweed derivatives containing alginic acid), which come as pads, ropes, and ribbons (AlgiSite, Sorbsan, Curasorb), are indicated for extensive exudate and for control of bleeding after surgical debridement. Foam dressings (Allevyn, LYOfoam, Hydrasorb, Mepilex, Curafoam, Contreet) are useful as they can handle a variety of levels of exudate and provide a moist environment for healing. Those with adhesive backings stay in place longer and need less frequent changing.

In some embodiments, a transdermal patch comprises permeation enhancer, e.g. transcutol, (diethylene glycol monoethyl ether), propylene glycol, dimethylsulfoxide (DMSO), menthol, 1-dodecylazepan-2-one (Azone), 2-nonyl-1,3-dioxolane (SEPA 009), sorbitan monolaurate (Span20), and dodecyl-2-dimethylaminopropanoate (DDAIP)., which may be provided at a weight/weight concentration of from about 0.1% to about 10%, usually from about 2.5% to about 7.5%, more usually about 5%.

Transdermal patches may further comprise additives to prevent crystallization. Such additives include, without limitation, one or more additives selected from octyldodecanol at a concentration of from about 1.5 to about 4% w/w of polymer; dextrin derivatives at a concentration of from about 2 to about 5% w/w of polymer; polyethylene glycol (PEG) at a concentration of from about 2 to about 5% w/w of polymer; polypropylene glycol (PPG) at a concentration of from about 2 to about 5% w/w of polymer; mannitol at a concentration of from about 2 to about 4% w/w of polymer; Poloxamer 407, 188, 401 and 402 at a concentration of from about 5 to about 10% w/w of polymer; and Poloxamines 904 and 908 at a concentration of from about 2 to about 6% w/w of polymer.

Polyvinylpyrrolidine (PVP) may also be included in a transdermal patch formulation, for example at a concentration of from about 5 wt % to about 25 weight%, about 7 wt % to about 20 wt %, about 8 wt % to about 18 wt %, about 10 wt % to about 16 wt %, about 10 wt %, about 12 wt %, about 14 wt %, about 16 wt %.

Emulsifiers which may be used include glyceryl stearate, polysorbate 60, PEG-6/PEG-32/glycol stearate mixture, etc. Solvents which may be used include the lower alcohols, in particular ethanol and isopropanol, and propylene glycol.

Hydrophilic gelling agents include carboxyvinyl polymers (carbomer), acrylic copolymers such as acrylate/alkylacrylate copolymers, polyacrylamides, polysaccharides, such as hydroxypropylcellulose, natural gums and clays, and, as lipophilic gelling agents, representative are the modified clays such as bentones, fatty acid metal salts such as aluminum stearates and hydrophobic silica, or ethylcellulose and polyethylene.

Therapeutic formulations for treatment of melanoma with blocker, e.g. Benzamil, can be used alone or in combination with an additional agent, e.g., a therapeutic agent, said additional agent being selected by the skilled artisan for its intended purpose. For example, the additional agent can be a therapeutic agent art-recognized as being useful to treat melanoma. The agents set forth below are illustrative and not intended to be limited. The combinations which are part of this invention can be a blocker and at least one additional agent selected from the lists below. The combination can also include more than one additional agent, e.g., two or three additional agents if the combination is such that the formed composition can perform its intended function.

Standard treatment for melanoma that may be combined with the administration of a blocker of the invention includes surgery; chemotherapy, e.g. one or more of taxanes, dacarbazine, temozolomide, melphalan, nab-paclitaxel, paclitaxel, carmustine, cisplatin, carboplatin, vinblastine, etc.; radiation therapy; biologic therapy, e.g. with one or more of TNFα, aldesleukin, dabrafenib, interferon alfa-2b, ipilimumab, trametinib, peginterferon alfa-2b, dabrafenib, vemurafenib, oncolytic virus, etc.

Suitable chemotherapeutic agents include, for example, platinum-based agents (such as carboplatin), vinca alkaloids, agents that disrupt microtubule formation, anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, anthracycline antibiotics, topoisomerase inhibitors, macrolides, therapeutic antibodies, retinoids; geldanamycin or a derivative thereof, and other standard chemotherapeutic agents well recognized in the art.

In some embodiments, a second agent is one of the following: a platinum-based agent (e.g., carboplatin or cisplatin), an anti-VEGF antibody (e.g., bevacizumab), dacarbazine or DTIC (also known as DIC, DTIC-Dome, or Imidazole Carboxamide), Oblimersen (or Genasense), interleukin-2 (IL-2), interferon (IFN), Interferon α-2b, a BRAF inhibitor (such as Vemurafenib (or Zelboraf), GDC-0879 (available from Tocris Bioscience), PLX-4720 (available from Symansis), or Sorafenib (or Sorafenib Tosylate or Nexavar (available from Bayer Pharmaceuticals Corp.,)), Dabrafenib (GSK2118436), LGX-818, CEP-32496, UI-152, RAF 265, Regorafenib (BAY 73-4506), or CCT239065), an antibody against the Programmed Death 1 (PD-1) receptor (such as BMS-936558, available from Bristol Myers Squibb), an antibody against the PD-1 Ligand (anti-PD-L1 antibody), or anti-CTLA-4 antibody such as Ipilimumab (or MDX-010, MDX-101, or Yervoy), or a DNA alkylating agent such as Temozolomide.

As will be understood by those of ordinary skill in the art, the appropriate doses of other agents will be approximately those already employed in clinical therapies wherein the other agent are administered alone or in combination with other agents. Variation in dosage will likely occur depending on the condition being treated. As described above, in some embodiments, the other agents may be administered at a reduced level.

A combination of the administration configurations described herein can be used. The combination therapy methods described herein may be performed alone or in conjunction with an additional therapy, such as chemotherapy, radiation therapy (e.g., whole brain radiation therapy), surgery, hormone therapy, gene therapy, immunotherapy, chemoimmunotherapy, cryotherapy, ultrasound therapy, local ablative therapy, radiofrequency ablation therapy, photodynamic therapy, and the like. Additionally, a person having a greater risk of developing melanoma may receive treatments to inhibit and/or delay the development of the disease.

The composition can be packaged in any suitable container to suit its viscosity and intended use. The invention accordingly also provides a closed container containing a therapeutically acceptable composition as herein defined.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### METHODS OF USE

A composition comprising an effective dose of an ENAC blocker, optionally combined with additional therapeutic agents, is provided to an individual with melanoma. The administration can be oral, parenteral, topical, etc. In some embodiments topical is preferred. The dosing and periodicity of administration is selected to provide for therapeutic efficacy.

The methods described herein may further comprise selecting patients for treatment (e.g., identifying an individual who is suitable for treatment for melanoma). Thus, for example, in some embodiments, a method described herein further comprises identifying the individual having one of the characteristics described herein, such as melanoma subtype or staging characteristics, LDH level, or BRAF status described herein. In some embodiments, there is provided a method of treating melanoma in an individual (e.g., human) comprising the steps of (a) determining whether the individual has melanoma such as a melanoma described herein, and (b) administering to the individual an effective amount of a composition of the invention.

In some embodiments, the method is used as a first line therapy. In some embodiments, the method is used as a second line therapy. In some embodiments, the individual has not received prior therapy (e.g., prior cytotoxic chemotherapy) for the melanoma (e.g., metastatic melanoma).

Melanoma described herein may be any of the following: cutaneous melanoma, extracutaneous melanoma, superficial spreading melanoma, malignant melanoma, nodular malignant melanoma, nodular melanoma, polypoid melanoma, acral lentiginous melanoma, lentiginous malignant melanoma, amelanotic melanoma, lentigo maligna melanoma, mucosal lentignous melanoma, mucosal melanoma, soft-tissue melanoma, ocular melanoma, desmoplastic melanoma, or metastatic malignant melanoma.

In some embodiments, the melanoma is melanoma of the skin. In some embodiments, the melanoma is superficial spreading melanoma. In some embodiments, the melanoma is nodular melanoma. In some embodiments, the melanoma is acral lentiginous melanoma. In some embodiments, the melanoma is lentigo maligna melanoma. In some embodiments, the melanoma is mucosal melanoma (e.g., mucosal melanoma in nose, mouth, throat, or genital area). In some embodiments, the melanoma is ocular melanoma. In some embodiments, the melanoma is uveal melanoma. In some embodiments, the melanoma is choroidal melanoma.

In some embodiments, the melanoma comprises a mutation in BRAF. In some embodiments, the melanoma comprises a BRAF V600E mutation. In some embodiments, the melanoma does not comprise a mutation in BRAF (e.g., the melanoma comprises wild-type BRAF). In some embodiments, the melanoma does not comprise BRAF mutant such as a BRAF mutant with increased activity (for example, increased kinase activity, and/or increased activity as compared to wild-type BRAF) or a BRAF gain-of-function mutant. In some embodiments, the melanoma does not comprise a constitutive active BRAF mutant. In some embodiments, the melanoma does not comprise BRAF V600E mutation (e.g., the melanoma comprises wild-type BRAF). In some embodiments, the melanoma comprises wild-type BRAF (e.g., the melanoma cells have wild-type BRAF). In some embodiments, the melanoma comprises a BRAF mutant such as a BRAF mutant with increased activity (for example, increased kinase activity, and/or increased activity as compared to wild-type BRAF) or a BRAF gain-of-function mutant.

In some embodiments, the individual has elevated serum lactate dehydrogenase (“LDH”) level. In some embodiments, the individual has serum LDH of less than about 0.8× upper limit of normal (“ULN”). In some embodiments, the individual has serum LDH at about 0.8× to about 1.1×ULN. In some embodiments, the individual has serum LDH of between greater than about 1.1× to about 2.0×ULN. In some embodiments, the individual has serum LDH of between about 1.1× to about 2.0×ULN.

In some embodiments, the individual is under about 65 years old. In some embodiments, the individual is at least about 65 years old (for example at least about any of 70, 75, or 80 years old). In some embodiments, the individual is a male. In some embodiments, the individual is a female. The individual may have at least one (e.g., at least any of 2, 3, 4, 5, 6, or 7) of the following characteristics: (1) Histologically or cytologically confirmed cutaneous malignant melanoma with evidence of metastasis (Stage IV); (2) No prior cytotoxic chemotherapy for metastatic malignant melanoma; (3) No prior adjuvant cytotoxic chemotherapy; (4) Male or non-pregnant and non-lactating female greater than 18 years of age; (5) No other current active malignancy within the past 3 years; (6) Radiographically-documented measurable disease (for example, the presence of at least 1 radiographically documented measurable lesion); and (7) ECOG performance status 0-1. In some embodiments, the individual does not have history or current evidence of brain metastases, including leptomeningeal involvement. In some embodiments, the individual does not have pre-existing peripheral neuropathy of NCI CTCAE Scale of Grade greater than 2.

In some embodiments, the melanoma to be treated is stage 0, stage I, stage II, stage III, or stage IV. In some embodiments, the melanoma to be treated is stage 0, stage IA, stage IB, stage IIA, stage IIB, stage IIC, stage IIIA, stage IIIB, stage IIIC, or stage IV. In some embodiments, the melanoma is metastatic melanoma. In some embodiments, the metastatic melanoma is at stage M1a. In some embodiments, the metastatic melanoma is at stage M1b. In some embodiments, the metastatic melanoma is at stage M1c. Staging of melanoma may be based on a method known to one skilled in the art. Staging of melanoma may be according to the criteria included in 2009 AJCC Melanoma Staging and Classification. See Balch C M et al., J Clin Oncol. 2009, 27(36):6199-206 (the contents disclosed therein are incorporated by reference in their entirety). For example, the staging of melanoma may be according to the criteria set forth in Tables 1 and 2.

In some embodiments, the individual has melanoma tumor with thickness of less than about any of 0.5 millimeter (“mm”), 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, or 8 mm. In some embodiments, the individual has melanoma tumor with thickness of at least about any of 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, or 8 mm. In some embodiments, the individual has melanoma tumor with thickness of about any of 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, or 8 mm. In some embodiments, the individual has melanoma tumor with thickness of about any of 0-1 mm, 1-2 mm, 2-3 mm, 3-4 mm, 4-5 mm, 5-6 mm, 1-4 mm, 1-6 mm, 2-4 mm, 2-6 mm, or 4-6 mm.

The methods provided herein may be practiced in an adjuvant setting. Adjuvant setting may refer to a clinical setting in which an individual has had a history of a cancer described herein, and generally (but not necessarily) been responsive to therapy, which includes, but is not limited to, surgery (e.g., surgery resection), radiotherapy, and chemotherapy; however, because of their history of cancer, these individuals are considered at risk of development of the disease. Treatment or administration in the adjuvant setting refers to a subsequent mode of treatment. The degree of risk (e.g., when an individual in the adjuvant setting is considered as “high risk” or “low risk”) depends upon several factors, most usually the extent of disease when first treated.

In some embodiments, the amount of the composition is sufficient to prolong progression-free survival of the individual. In some embodiments, the amount of the composition is sufficient to prolong survival of the individual. In some embodiments, the amount of the composition is sufficient to improve quality of life of the individual. In some embodiments, the amount of the composition (for example when administered alone) is sufficient to produce clinical benefit of more than about any of 50%, 60%, 70%, or 77% among a population of individuals treated by the methods of the invention.

In some embodiments, the amount of the composition, first therapy, second therapy, or combination therapy is an amount sufficient to decrease the size of a melanoma tumor, decrease the number of melanoma tumor cells, or decrease the growth rate of a melanoma tumor by at least about any of 10%, 20%, 30%, 40%, 50%, 60%, 70%, 80%, 90%, 95% or 100% compared to the corresponding tumor size, number of melanoma tumor cells, or tumor growth rate in the same individual prior to treatment or compared to the corresponding activity in other individuals not receiving the treatment. Methods that can be used to measure the magnitude of this effect are known in the field.

In some embodiments, the amount of the blocker, e.g. Benzamil, in the composition is included in any of the following ranges: about 0.1 mg to about 500 mg, about 0.1 mg to about 2.5 mg, about 0.5 to about 5 mg, about 5 to about 10 mg, about 10 to about 15 mg, about 15 to about 20 mg, about 20 to about 25 mg, about 20 to about 50 mg, about 25 to about 50 mg, about 50 to about 75 mg, about 50 to about 100 mg, about 75 to about 100 mg, about 100 to about 125 mg, about 125 to about 150 mg, about 150 to about 175 mg, about 175 to about 200 mg, about 200 to about 225 mg, about 225 to about 250 mg, about 250 to about 300 mg, about 300 to about 350 mg, about 350 to about 400 mg, about 400 to about 450 mg, or about 450 to about 500 mg.

Exemplary effective amounts include, but are not limited to, at least about any of 10 mg/m² to about 1080 mg/m² of the blocker; at least about 30 mg/m², at least about 50 mg/m², at least about 60 mg/m², at least about 75 mg/m², at least about 80 mg/m², at least about 90 mg/m², at least about 100 mg/m², at least about 120 mg/m², at least about 125 mg/m², at least about 150 mg/m², at least about 160 mg/m², at least about 175 mg/m², at least about 180 mg/m², at least about 200 mg/m², at least about 210 mg/m², at least about 220 mg/m², at least about 250 mg/m², at least about 260 mg/m², at least about 300 mg/m², at least about 350 mg/m², at least about 400 mg/m², at least about 500 mg/m², at least about 540 mg/m², up to about 750 mg/m², up to about 1000 mg/m², or up to about 1080 mg/m² of the blocker, e.g. benzamil. In some embodiments of any of the above aspects, the effective amount in the composition includes at least about any of 0.1 mg/kg, 0.5 mg/kg, 1 mg/kg, 5 mg/kg, 7.5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, or 100 mg/kg. In various embodiments, the effective amount of the blocker, e.g. benzamil in the composition includes less than about any of 350 mg/kg, 300 mg/kg, 250 mg/kg, 200 mg/kg, 150 mg/kg, 100 mg/kg, 50 mg/kg, 25 mg/kg, 20 mg/kg, 10 mg/kg, 7.5 mg/kg, 6.5 mg/kg, 5 mg/kg, 3.5 mg/kg, 2.5 mg/kg, or 1 mg/kg.

Exemplary dosing frequencies include, but are not limited to, daily, every two days, every three days, every four days, every five days, every six days, weekly without break, weekly for three out of four weeks, once every three weeks, once every two weeks, or two out of three weeks. In some embodiments, the composition is administered about once every 2 weeks, once every 3 weeks, once every 4 weeks, once every 6 weeks, or once every 8 weeks. In some embodiments, the composition is administered at least about any of 1×, 2×, 3×, 4×, 5×, 6×, or 7× (i.e., daily) a week. In some embodiments, the intervals between each administration are less than about any of 6 months, 3 months, 1 month, 20 days, 15, days, 14 days, 13 days, 12 days, 11 days, 10 days, 9 days, 8 days, 7 days, 6 days, 5 days, 4 days, 3 days, 2 days, or 1 day. In some embodiments, the intervals between each administration are more than about any of 1 month, 2 months, 3 months, 4 months, 5 months, 6 months, 8 months, or 12 months. In some embodiments, there is no break in the dosing schedule. In some embodiments, the interval between each administration is no more than about a week.

### EXAMPLES

The following examples are offered by way of illustration and not by way of limitation.

In a matrigel invasion assay (48 hours, DMEM, 10% FBS as chemo attractant), V12 Rac1 mutant primary foreskin-derived melanocytes significantly invaded compared to LacZ control. Invasion was abolished in conditions treated with 50 μM Benzamil. Dosage up to 100 uM were tolerated in vitro (assessed by detectable proliferation), although proliferation-rate was significantly reduced at 50 and 100 μM, (absorbance 570 nanometer; MTT proliferation assay, ATCC, Manassas, US) with and without Benzamil (cultured in DMEM, 10% FBS, 1% antibiotic/antimycotic).

Benzamil reduce invasion and lung metastasis in vivo in a xenograft model. V12 Rac1 or LacZ control primary foreskin-derived melanocytes were seeded together with autologous primary keratinocytes ratio 1:4 or 1:10 onto devitalized dermis. After 6 days of culture in KGM media, 10% FBS, 1% antibiotic/antimycotic, skin equivalents were grafted to NOD/SCID gamma mice (n=6). After 10 days sutures and bandages were removed (=day 0), and V12 Rac1 xenografted animals injected IP with Benzamil (5 mg/kg) or PBS every other day for 20 days. Grafts and lungs were harvested on day 21. V12 Rac1 melanocytes displayed primary invasion from the skin (HMB45+ cells sub-GoH3, assayed by confocal IF), distinct from LacZ and significantly more than V12 Rac1 grafts from Benzamil-treated animals. Moreover, V12 Rac1 grafted animals had multiple foci of HMB45+ cells in lungs, distinct from control and significantly more than Benzamil treated animals.

Benzamil reduce invasion and proliferation of melanoma cell lines expressing increased Rac1GTP. Melanoma cell lines COLO829, MM485 and CHL1 displayed increased Rac1GTP (Rac1GTP mAb, confocal IF), and 24 hours treatment with Benzamil (50 μM) redistributed Rac1GTP subcellular localization. Benzamil (50 μM) significantly reduced invasion in matrigel invasion assays of COLO829,MM485,CHL1. Invasion and proliferation was reduced more than PLX-4032 for cell lines MM485 and CHL1 (non-BRAF V600E mutant cell lines), and comparable to a Rac1 inhibitor (Rac1 inhibitor #553502, Calbiochem). Stimulation with Benzamil at 50 μM was not toxic, but reduced proliferation in COLO829,MM485,CHL1 melanoma cell lines similar to V12 Rac1 mutant primary melanocytes.

Reduction of NFKB (PRELA) and STAT3 (PSTAT3) signaling following Benzamil treatment in vivo and in vitro. Skin from V12 Rac1, control or Benzamil treated V12 Rac1 xenografted animals were assayed by confocal IF for phospho-Rela (upper) and phospo-STAT3 (lower panel) co-localizing with HMB45+ melanocytes. Both NFKB and STAT3 activation in HMB45+ cells was reduced in skin following benzamil treatment (5 mg/kg; 10 injections, EOD). Nuclear translocation of activated STAT3 and RelA in melanoma cell lines CHL1, COLO829 and MM485 was assayed after 24 hours by confocal IF of methanol-fixed cells on collagen coated coverslips, with and without exposure to Benzamil (50 μM). STAT3 and NFKB activation was diminished following Benzamil treatment for 24 hours in all three cell lines.

### Material and Methods

Isolation of primary melanocytes and culture of melanoma cell lines. Neonatal foreskin was incubated overnight at 4C in HBSS with 25 Wm! dispase. Epidermis-dermis was separated with foreceps, epidermal sheets trypsinized for 15 min, neutralized with DMEM containing 10% FBS, 1% AA, centrifuged for 5 min at 1000 rpm, and resuspended in medium 154 (Invitrogen, M-254-500) supplemented with 1% Human melanocyte growth supplement (Invitrogen, S-002-5) and 1% antibiotic-antimycotic. Melanoma cell lines CHL-1, Colo829, and MM485 were obtained from ATCC. Colo829 and MM485 were cultured in grow in RPMI (ATCC) with 10% fetal bovine serum (FBS), 1% antibiotic/antimycotic and CHL1 in DMEM (Mediatech Inc) with 10% FBS and 1% antibiotic/antimycotic.

V12 Rac1 mutant overexpression. V12 Rac1 or LacZ retrovirus was produced from V12 or LacZ DNX cells and cultured at 37C in DMEM with 10% FBS 1% AA on 15 cm plates containing 30 ml media. At 80% confluency, plates were transferred to 32C, and media collected after 24 and 48 hours. For each subsequent infection, fresh virus media was kept on ice, while primary melanocytes were pretreated with 10 ul polybrene (5 mg/ml) per 10 ml dish and incubated for 10 minutes at 37C. 10 ul (5 mg/ml) polybrene was subsequently added to each 10 ml aliquote of virus media, and added to each melanocyte-dish after aspirating existing media. Plates were centrifuged at 1000 rpm at 32C for 1 hour, whereafter dishes were placed in a 37C incubator for 4 hours, before changing media to supplemented media 154, as described above. Virus efficacy was verified by expression of its myc-tag by western blot of cell lysates of one dish per batch.

Drug concentrations and dilutions. Benzamil hydrochloride hydrate was dissolved in 25 ml of ddH2O for a working concentration of 2 mg/ml. For in vivo applications, 5 mg/kg and 10 mg/kg, and in vitro 1-100 uM. Rac1 inhibitor 553502, Calbiochem, US, was dissolved in ddH2O to a stock solution of 100 uM, and 75 uM was used for in vitro studies. PLX-4032 (Selleckchem) was dissolved to a 1 uM in DMSO stock solution.

Rac1-GTP-phosphorylated-STAT3 and phosphorylated-P65 assays. For assaying tissue sections, tissue was embedded in OCT, snap frozen, and 7 uM sections cut on a cryostat (Leica). Sections were fixed for 10 minutes in cold methanol, washed once with TBS, then blocked for one hour at room-temp with 10% normal goat serum, and incubated with Rac1GTP 1:2000 in PBS (NewEast biosciences, US phosphor-STAT3 1:200 (Cell Signaling) or phosphor-P65 (Cell Signaling, US) 1:200 in PBS. Sections were washed and incubated with secondary antibodies 1:400 together with Hoescht 1:5000 for 1 hour at room temperature, and washed and mounted with fluoromount (Southern Biotech, US). For in vitro studies, 20 000 melanocytes were seeded onto collagen-coated coverslips in 6-well plates. After attaching, cells were either grown in the presence or absence of growth factors (HMGS) and FBS. After 24 hours, plates were washed with cold TBS and fixed with 100% cold methanol for 10 minutes, prior to antibody incubation as described above. Mounted slides were imaged with using confocal microscopy (LSM-700, Zeiss, Germany).

Matrigel invasion assay. Wells and inserts of 8 um BioCoat Matrigel Invasion Chambers, (BD Biosystems) were rehydrated for 2 hours at 37C, 5% CO2. For each insert, 100 000 cells were serum starved for 24 hours at 37C, trypsinised, pelleted and ressupended in 500 ul DMEM. 750 ul per well of DMEM with 10% fetal bovine serum was used as chemoattractant. Wells and inserts were incubated at 37C for 48 hours, whereby media was aspirated, and inserts fixed in 4% paraformaldehyde in PBS for 30 min. Each Insert was washed x3 with tap water, and incubated in 1% crystal violet for 3 hours. Finally, inserts were rinsed in tap water x3, top side of membranes were scrubbed with a cotton tip scrub, and dried overnight.

MTT proliferation assay. 25000 melanocytes were plated in each well in a 96 well plate, and incubated with 100 ul DMEM with 10% FBS 1% AA for 24 hours. 10 ul MTT reagent (MTT proliferation assay, ATCC, Manassas, US) was added for 4 hours, 100 ul detergent was added to each well for 2 hours., and absorbance read at 570 nm (Spectramax M5, Molecular Devices, US), normalized to cell-free control absorbance.

Xenografting of organotypic skin equivalents. V12 Rac1 or LacZ control primary foreskin-derived melanocytes were seeded together with autologous primary keratinocytes ratio 1:4 or 1:10 onto devitalized dermis (NY Firefighters biobank, NY, US). After 6 days of culture in KGM media, 10% FBS, 1% antibiotic/antimycotic, skin equivalents were grafted to NOD/SCID gamma mice (n=6). After 10 days sutures and bandages were removed, and V12 Rac1 xenografted animals injected IP with Benzamil (5 mg/kg) or PBS every other day for 20 days. Grafts and lungs were harvested on day 21.

What is claimed is:

1. A method of treating melanoma in a subject, the method comprising:
administering to the subject an effective dose of a blocker of a benzamil sensitive protein for duration and periodicity sufficient to reduce the symptoms of melanoma.

2. The method of claim 1, wherein the blocker is amiloride or a derivative thereof.

3. The method of claim 2, wherein the blocker is benzyl amiloride.

4. The method of claim 1, wherein the administration is systemic.

5. The method of claim 1, wherein the administration is topical.

6. The method of claim 1, wherein the subject is a human.

7. The method of claim 3, wherein the effective dose is from about 10 mg/m² to about 1080 mg/m².

8. The method of claim 1, further comprising administering an effective dose of a second agent effective in the treatment of melanoma.

9. The method of claim 1, wherein treatment inhibits tumor growth.

10. The method of claim 1, wherein the treatment inhibits metastasis.

11. A composition comprising an effective dose of a blocker of a benzamil sensitive protein for use in the methods of any one of claims 1-10.

12. The composition of claim 11, provided in a unit dose formulation.
